(19) United States
(12) Patent Application Publication
(10) Pub. No.: US 2009/0259262 A1
(43) Pub. Date: Oct. 15, 2009

(54) SURGICAL TOOL

(75) Inventor: Jerome NAYET, Saint Genis Pouilly (FR)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/270,167

(22) Filed: Nov. 13, 2008

(30) Foreign Application Priority Data
Apr. 15, 2008 (FR) 0852526

(51) Int. Cl. A61B 17/88

(52) U.S. Cl. 606/86 A

(57) ABSTRACT

Provided herein is a surgical tool for performing a distraction or compression procedure that includes a first member and a second member pivotally connected together at a pivot point and configured to be moved between a first position and a second position about a common axis defined by the pivot point. The surgical tool further includes a first leg connected to the first member and a second leg connected to the second member, the first leg and the second leg extending from the pivot point and terminating at respective distal ends, wherein the first leg comprises a first portion that is displaceable between a first position and a second position independent of the position of the first member and the second member.

### BACKGROUND

1. Field of the Disclosure

This disclosure is directed to a surgical tool for manipulating an implant, and more particularly a surgical tool for displacement of an implant along a rod.

2. Description of the Related Art

Various forms of the spinal abnormalities exist, including for example ruptured disks or curvatures of the spine. Given the various abnormalities, various implants and techniques to treat such abnormalities exist. In the particular context of spinal curvatures, including lordosis or kyphosis, one particular method of correcting such curvatures can include implanting a rod and anchor system to re-align the spine. Oftentimes, such a medical procedure includes the implantation of hooks or anchors, such as screws within portions of the spine which are then attached to a rod. A distraction or compression procedure can then be undertaken in which the implanted anchors, which are lodged within the vertebrate, are changed in position along the rod, thereby extending or compressing a portion of the spine to more closely align with the rod, and a more ideal spinal position.

Currently, such distraction and compression procedures typically utilize multiple tools. In fact, it is commonplace for a surgeon to use both hands to complete the such processes, including one hand on a vise grip tool, intended to grip the rod, and another hand using pliers that are placed between the vise grip and the implant. During the procedure, the surgeon holds the vise grip and engages the pliers to move the implant relative to the vice grip thereby distracting or compressing the implants and correcting the curvature of the spine.

### SUMMARY

According to a first aspect, a surgical tool for manipulating an implant is provided that includes a first member and a second member pivotally connected together at a pivot point and configured to be moved between a first position and a second position about a common axis defined by the pivot point. The surgical tool further includes a first leg connected to the first member and a second leg connected to the second member, wherein the first leg and the second leg extend from the pivot point and terminate at respective distal ends. The first leg includes a first portion that is displaceable between a first position and a second position independent of the position of the first member and the second member. In one embodiment of the first aspect, a locking member is connected to the first member and extends between the first member and the second member to lock the position of the first member relative to the second member during movement between the first position and second position. In another embodiment, a spring extends between the first member and the second member and biases the first member and the second member against each other.

In still another embodiment of the first aspect, the first leg includes an articulating joint between the distal end and the pivot point. In a more particular embodiment, the articulating joint is a hinge, including a pin extending through an opening in the first portion. In still another particular embodiment, the first portion is between the articulating joint and the distal end, and the first portion is configured to articulate around a single axis of the articulating joint in a direction towards the second leg.

In another embodiment the first leg further includes a second portion between the articulating joint and the pivot point. According to one embodiment of the first aspect, the second portion is configured to articulate around a single axis of the articulating joint in a direction away from the second leg. In another embodiment of the first aspect, the first portion is displaceable between the first position and the second position at a displacement angle relative to the first portion and a second portion within the first leg of not greater than about 90°.

As such, in another particular embodiment, the second portion has a length that is not greater than about 80% of a length of the first leg. According to a more particular embodiment, the second portion has a length that is not greater than the length of the first portion. In another embodiment of the first aspect, the first portion has a length that is not greater than about 80% of a length of the first leg. In a more particular embodiment, the first portion has a length that is within a range between about 10% and about 70% of the length of the first leg. Still, according to another embodiment, the second leg is displaceable between a first position and a second position dependent upon the position of the first member and the second member.

According to another embodiment of the first aspect of the present disclosure, the surgical tool further includes a first head connected to the first portion adjacent to the distal end of the first leg and configured to engage an implant, and a second head connected to the second leg adjacent to the distal end of the second leg and configured to engage the implant. In a more particular embodiment, the first head comprises a first channel and the second head comprises a second channel. In another particular embodiment, the first channel has a width that is less than a width of the second channel. In a still more particular embodiment, the width of the first channel is less than the width of the second channel by at least about 3% of the width of the second channel. According to one particular embodiment, the width of the first channel is less than the width of the second channel within a range between about 3% and about 60% of the width of the second channel.

In another embodiment of the first aspect, the first channel has a width and is configured to engage a rod having a diameter, wherein the width of the first channel is greater than the diameter of the rod by not greater than about 20% of the diameter of the rod. Accordingly, in another embodiment, the width of the first channel is greater than the diameter of the rod within a range between 3% and about 10% of the diameter of the rod. Still, in one particular embodiment, the first channel has a width of at least about 3 mm. In one certain embodiment, the width of the first channel is within a range between about 3 mm and about 10 mm.

According to another particular embodiment of the first aspect, the second channel has a width and is configured to slidably engage a rod having a diameter, wherein the width of the second channel is greater than the diameter of the rod by at least about 5% of the diameter of the rod. Accordingly, in one embodiment, the width of the second channel is greater than the diameter of the rod by not greater than about 60% of the diameter of the rod. As such, in another embodiment, the second channel has a width of at least about 5 mm.

In a distinct embodiment of the first aspect, the first portion comprises a quick-connect coupling. In a more particular embodiment, the quick-connect coupling is configured to engage a plurality of interchangeable heads attachable to the first portion. In a still more particular embodiment, each of the plurality of interchangeable heads comprise a channel configured to engage a rod, wherein each channel of each interchangeable head has a channel width that is different than a channel width of a different interchangeable head.

According to a second aspect, a surgical tool configured to complete a distraction or compression procedure includes a first member and a second member pivotally connected together at a pivot point and configured to be moved between a first position and a second position about a common axis defined by the pivot point. The surgical tool further includes a first leg connected to the first member and extending from the pivot point to a distal end, and a second leg connected to the second member and extending from the pivot point to a distal end. A first portion of the first leg is configured to engage a rod and fix the axial position of the first portion with respect to the rod during movement of the first member and second member between the first position and the second position. In one embodiment, the second leg is configured to slidably communicate with the rod during movement of the first member and second member between the first position and the second position. In another embodiment, the second leg is displaced in a direction along the rod away from the first leg upon movement of the first member and second member between the first position and the second position.

In one particular embodiment of the second aspect, the first portion is configured to frictionally engage the rod and fix the position of the first portion relative to the rod by exerting a torsional force on the rod. In another embodiment, the first member, second member, first leg, and second leg comprise an autoclavable material.

In another aspect, a surgical tool includes a first member and a second member pivotally connected together at a pivot point. The first member includes a proximal end and a distal end, wherein the pivot point is between the proximal end and the distal end, and a first channel between the distal end and pivot point. The second member includes a proximal end and a distal end, wherein the pivot point is between the proximal end and the distal end, and a second channel between the pivot point and the distal end, wherein the second channel has a width that is greater than a width of the first channel. As such, in accordance with one embodiment, the width of the second channel is at least about 0.3 mm greater than the width of the first channel. In accordance with another embodiment, the width of the second channel is within a range between about 0.3 mm and about 5 mm greater than the width of the first channel.

In still another aspect, a surgical tool for performing a distraction or compression of implants includes a first member having a first proximal portion between a proximal end and a pivot point and a first distal portion between a distal end and the pivot point. The surgical tool further includes a second member pivotally connected to the first member at the pivot point and having a second proximal portion between a proximal end and a pivot point and a second distal portion between a distal end and the pivot point, wherein the second distal portion comprises at least one more articulating joint than the first distal portion.

### BRIEF DESCRIPTION OF THE DRAWINGS

FIG. 1 includes a lateral view of a portion of a vertebral column.

FIG. 2 includes a rearview of a portion of a spinal column having an abnormal curvature.

FIG. 3 includes an illustration of a surgical tool in accordance with one embodiment.

FIG. 4 includes an illustration of a surgical tool in accordance with one embodiment.

FIG. 5 includes an illustration of a top view of a portion of a surgical tool in accordance with one embodiment.

FIG. 6 includes a side view of the portion of the surgical tool of FIG. 5 in accordance with one embodiment.

FIG. 7 includes a side view of a portion of a surgical tool in accordance with one embodiment.

FIG. 8 includes a side view of the portion of the surgical tool of FIG. 7 in accordance with one embodiment.

FIG. 9 includes a side view of a head portion of a member of a surgical tool in accordance with one embodiment.

FIG. 10 includes a side view of a head portion of a member of a surgical tool in accordance with one embodiment.

FIG. 11 includes an illustration of a surgical tool during a distraction or compression procedure in accordance with one embodiment.

FIG. 12 includes an illustration of a surgical tool in accordance with an embodiment.

### DESCRIPTION OF THE EMBODIMENT(S)

### Description of Relevant Anatomy

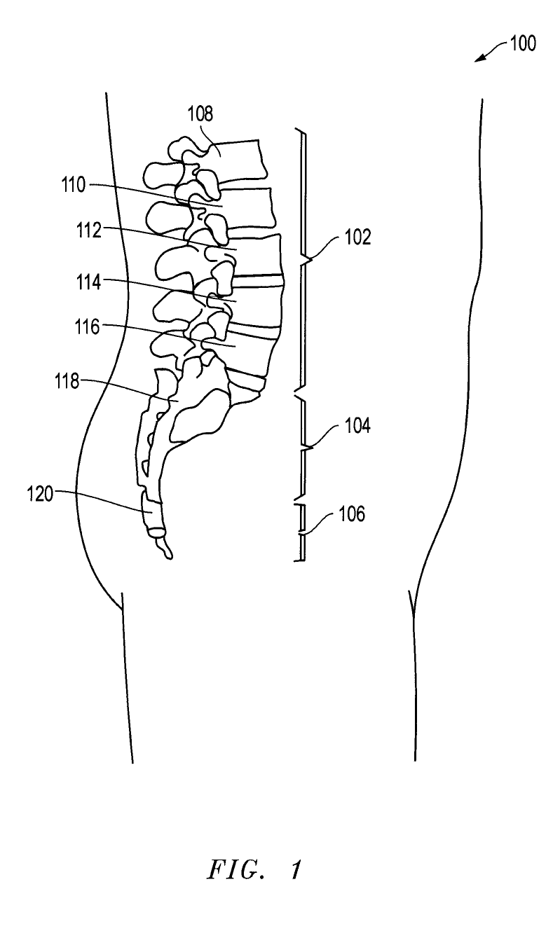

Referring initially to FIG. 1, a portion of a vertebral column, designated 100, is shown. As depicted, the vertebral column 100 includes a lumbar region 102, a sacral region 104, and a coccygeal region 106. The vertebral column 100 also includes a cervical region and a thoracic region. For clarity and ease of discussion, the cervical region and the thoracic region are not illustrated.

As illustrated in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

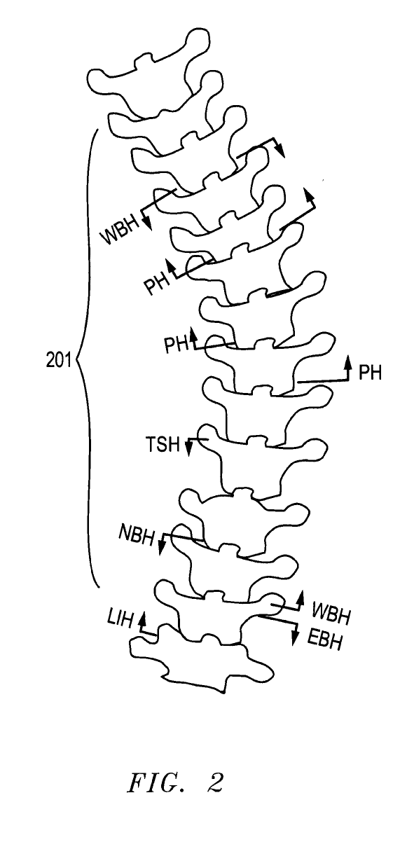

Referring to FIG. 2, a back view of a portion of a spine having an abnormal curvature is illustrated. Generally, such a curvature is commonplace with diseases such as lordosis or kyphosis. For example, a distraction of the spine within region 201 may be undertaken to correct the abnormal curvature. Such a distraction procedure within region 201 may include the implantation of anchors within portions of the spine, such as within the pedicles. The anchors in turn can be connected to a rod which spans the spine within region 201 or possibly over a greater region depending upon the surgeon's preference, and the necessary degree of correction. In fact, depending upon the severity of the curvature, the surgeon may opt to use rod and screw systems in multiple locations, for example, on both sides of the spine. During implantation of the rod and screw system, the curvature can be changed via a distraction or compression procedure, moving the spine toward a more natural spinal state. Periodically, the patient can undergo further procedures in which the surgeon changes the position of the implants relative to the rod thereby straightening the spine. Such a process can include the use of particular surgical tools, one of which can include distraction/compression forceps.

### Description of the Surgical Tool

The surgical tool described herein and its various embodiments facilitates in-situ (i.e., within a patient) manipulation of implants, and more particularly, single-handed manipulation of an implant. Generally, the surgical tool can engage an implant such as a rod and be used to induce translational displacement of another implant coupled to the rod, such as a screw, hook or the like such that upon operation by the user, the position of the implant with respect to the rod is changed. Accordingly, the surgical tool described herein is well suited for completing distraction and/or compression procedures.

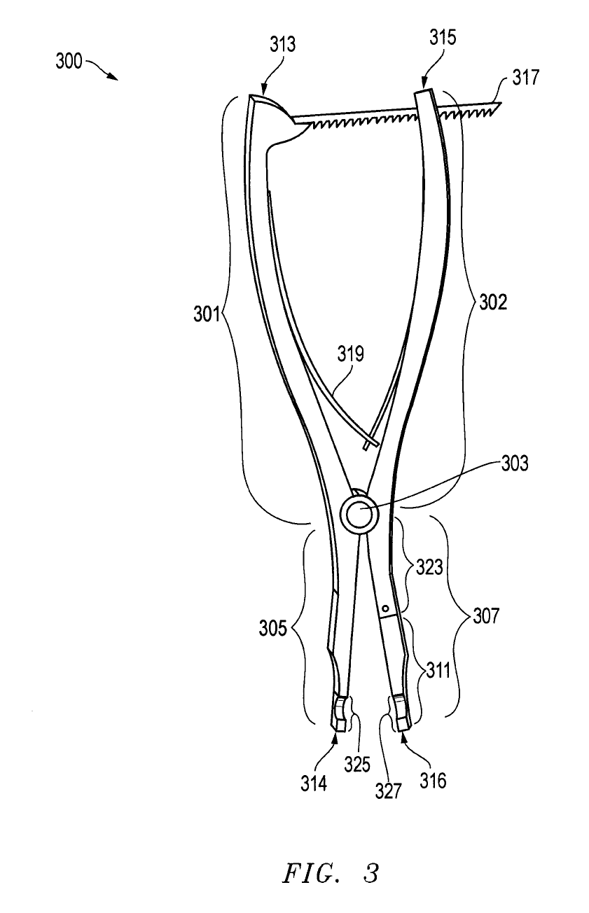

Referring to FIG. 3, a surgical tool is illustrated in accordance with one embodiment. The tool 300 includes a member 301 and a member 302 (i.e., handles) pivotally connected together at a pivot point 303 such that the members 301 and 302 can be moved between a first position and second position about a common axis defined by the pivot point 303. As illustrated, the member 301 extends between a proximal end 313 and the pivot point 303, and the member 302 extends between a proximal end 315 and the pivot point 303.

The tool 300 further includes a locking member 317. According to one embodiment, the locking member 317 is pivotally connected to the member 301 near the proximal end 313. The locking member 317 can extend between the members 301 and 302 during operation of the distraction/compression forceps. In one embodiment, the locking member 317 includes teeth along a bottom surface such that during a distraction or compression procedure, the members 301 and 302 are moved closer to each other and the locking member 317 is capable of fixing the position of the members 301 and 302 relative to each other.

The tool 300 further include a spring 319 extending between the members 301 and 302 and biasing the member 301 against the member 302. The spring 319 facilitates inadvertent movement of the members 301 and 302 towards each other without operator intervention.

The tool 300 further includes a leg portion 305 connected to the member 301 extending from the pivot point 303 and ending at a distal end 314. Likewise, member 302 is connected to a leg portion 307 that extends from the pivot point 303 to a distal end 316. According to one embodiment, the members 301 and 302 can be fixably attached to leg portions 305 and 307 respectively, such that they are a unitary or monolithic piece of material.

The leg portion 305 includes a head portion 325 connected to the leg portion 305 and adjacent to the distal end 314 of the leg portion 305. According to one embodiment the head portion 325 is configured to engage an implant. In a more particular embodiment, the head portion 325 includes a channel extending in a direction substantially orthogonal to the length of the leg portion 305 and configured to engage an implant such as a rod. Particular features of the head portion 325 will be illustrated in more detail in the following figures.

Leg portion 307, and more particularly the upper portion 311, also includes a head portion 327 connected to the upper portion 311 and adjacent to the distal end 316. Like the head portion 325, the head portion 327 is configured to engage an implant. According to one particular embodiment, the head portion 327 includes a channel extending substantially orthogonal to the length of the upper portion 311 and configured to engage an implant such as a rod.

According to one embodiment, leg portion 307 includes an articulating joint 321 between the pivot point 303 and the distal end 316. According to a particular embodiment, the articulating joint 321 can be a hinge, such that it includes an opening and a pin extending through the opening. As illustrated, the leg portion 307 includes a lower portion 325 between the pivot point 303 and the articulating joint 321 and an upper portion 311 disposed between the articulating joint 321 and the distal end 316.

The articulating joint 321 facilitates the pivoting of upper portion 311 relative to the lower portion 323 of the leg portion 307. As such, the upper portion 311 is configured to articulate around the articulating joint 321 in a direction toward the leg portion 305. In one embodiment, the upper portion 311 is displaceable between a first position and a second position independent of the position of the members 301 and 302.

Referring briefly to FIG. 4, the independent movement of the upper portion 311 is more clearly illustrated. FIG. 4 illustrates the tool of FIG. 3, including the members 301 and 302 in the same position relative to each other, however, the upper portion 311 has articulated around the articulating joint 321 relative to the lower portion 323 independent of the members 301 and 302. As further illustrated in FIG. 4, the upper portion 311 articulates in a direction towards the leg portion 305. According to one embodiment, the upper portion 311 only articulates in a direction toward the leg portion 305 and may not articulate in a direction away from the leg portion 305 beyond that of its original starting position illustrated in FIG. 3. In a more particular embodiment, the upper portion 311 is capable of displacement at an angle measured between the lower portion 325 and the upper portion 311 of at least about 5°. In another embodiment, the angle of displacement can be greater, such as at least about 10°, at least about 30°, at least about 60°, or even at least about 90°. Generally, the displacement angle is limited by the lower portion 325 and as such is not greater than about 180°.

The independent motion of the upper portion 311 within the leg portion 307 facilitates a locking position of the upper portion 311 relative to an implant, such as a rod engaged by the tool. Notably, in one embodiment, while the movement of the upper portion 311 is independent of the movement of the members 301 and 302, the leg portion 305 is dependent upon the movement of members 301 and 302.

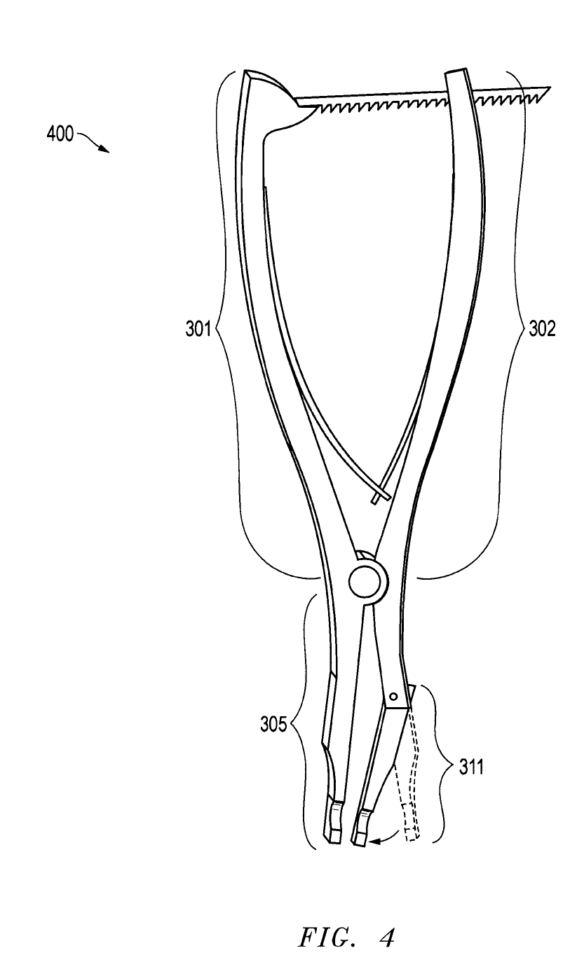

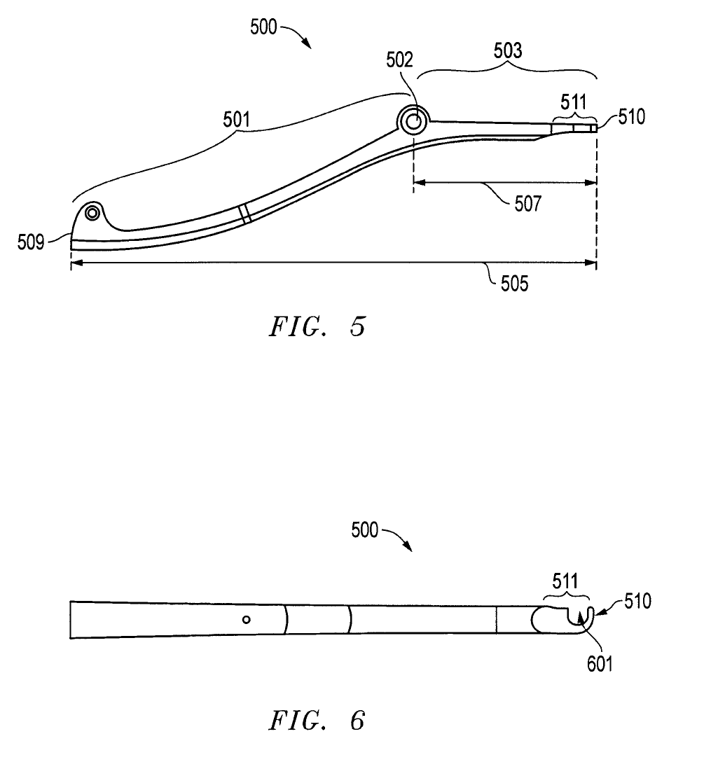

FIG. 5 includes a top view of a portion of the surgical tool in accordance with one embodiment. The portion 500 includes a member 501 extending between a proximal end 509 and a pivot point 502, and a leg portion 503 connected to the member 501 and extending from the pivot point 502 to a distal end 510. The portion 500 of the tool further includes a head portion 511 within the leg portion 503 adjacent to the distal end 510. According to one particular embodiment, the member 501 and leg portion 503 are a single unitary piece formed from the same material.

Generally, the lengths of the member 501 and leg portion 503 make up the entire length 505 of the tool. According to one embodiment, the length 505 is not greater than about 50 cm. In another embodiment, the length 505 can be less such as not greater than about 45 cm, or not greater than about 40 cm, or even not greater than about 35 cm. Still in another embodiment, the length 505 is at least about 20 cm, such as at least about 25 cm. In one particular embodiment, the length 505 is within a range between about 25 cm and about 35 cm.

As illustrated in FIG. 5, the length 507 of the leg portion 503 is a fraction of the length 505 of the tool, generally not greater than 50% of the length 505 of the tool. According to an embodiment, the length 507 of the leg portion 503 is not greater than about 45%, such as not greater than about 40%, or even not greater than about 30% of the length 505 of the tool. Generally, the length 507 of the leg portion 503 is at least about 10%, such as at least about 15%, or even at least about 20% of the length 505 of the tool. In a particular embodiment, the length 507 of the leg portion 503 is within a range between about 20% and about 30% of the length 505 of the tool.

In reference to actual values, in one embodiment, the leg portion 503 has a length 507 that is not greater than about 25 cm. According to another embodiment, the length 507 of the leg portion 503 is not greater than about 20 cm, such as not greater than about 15 cm, or not greater than about 10 cm. Generally, the length of leg portion 503 is within a range between about 4 cm and 10 cm, and more particularly within a range between about 6 cm and about 8 cm.

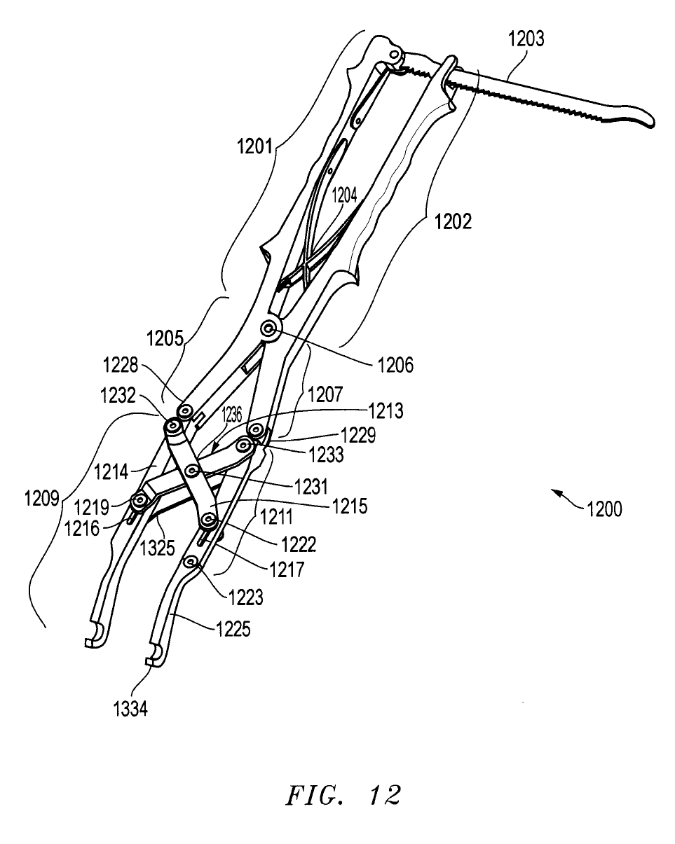

FIG. 6 includes a side view of the portion 500 of the tool illustrated in FIG. 5 in accordance with one embodiment. In particular, FIG. 6 more clearly illustrates the head portion 511. As illustrated, in one particular embodiment, the head portion 511 includes the channel 601 adjacent to the distal end 510 and configured to engage an implant, such as a rod. During a distraction or compression procedure, the channel 601 can be coupled to a rod, such that it slidably engages the rod, thus facilitating a distraction or compression procedure as will be more clearly described in accordance with FIG. 11.

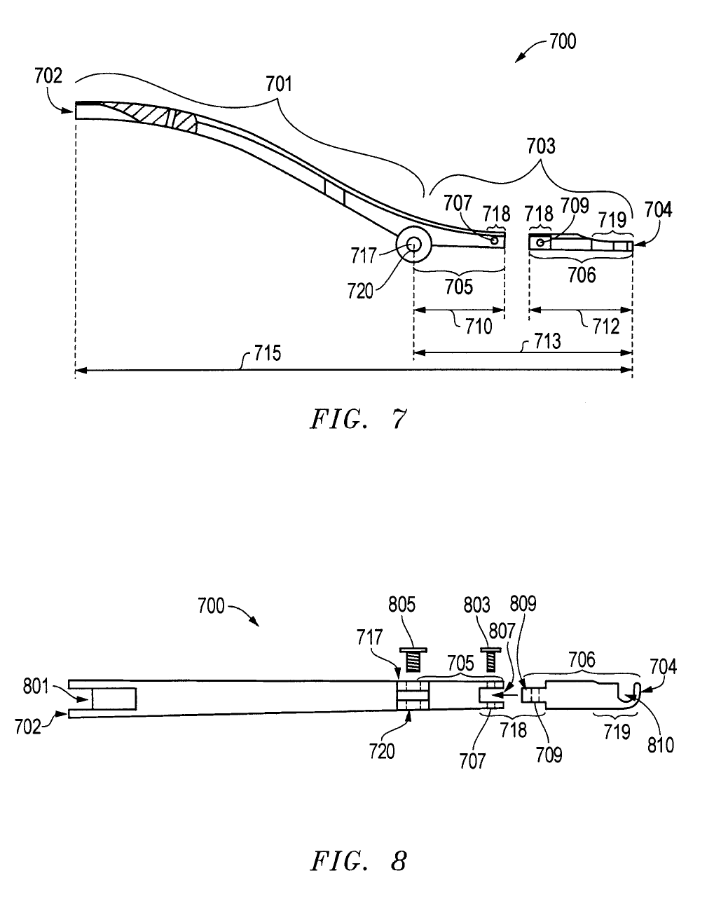

Referring to FIG. 7, a top view of a portion of the surgical tool in accordance with one embodiment, is illustrated. The portion 700 can be coupled to the portion 500 illustrated in FIG. 5 at a pivot point 717, such that the portions 700 and 500 can pivot around a common axis. The portion 700 includes a member 701 between a proximal end 702 and the pivot point 717 and a leg portion 703 connected to the member 701 and extending from the pivot point 717 to a distal end 704. The pivot point 717 can include a fastener to facilitate coupling of the members. Moreover, the length 715 of the portion 700 of the tool (i.e., the lengths of the member 701 and the leg portion 703) is the same as previously described in accordance with the portion 500 provided in FIG. 5. Additionally, the length 713 of the leg portion 703 relative to the length 715 of the portion 700 is the same as the length 507 described in accordance with FIG. 5.

The portion 700 includes a leg portion 703 having two separate and distinct portions, notably a lower portion 705 and an upper portion 706. According to one particular embodiment, the lower portion 705 and upper portion 706 are connected together by an articulating joint 718. In one particular embodiment, the articulating joint is a hinge, including a pin extending through openings 707 and 709 of the lower portion 705 and upper portion 706 respectively.

According to one embodiment, the lower portion 705 has a length 710 that is a fraction of the length 713 of the leg portion 703. The length 710 of the lower portion 705 is measured between the center of the pivot pint 717 to the end of the portion at the articulating joint 718. In one embodiment, the length 710 of the lower portion 705 is not greater than about 80% of the length 713 of the leg portion 703. In another embodiment, the length 710 is not greater than about 70%, such is not greater than about 60%, or even not greater than about 50% of the length 713 of the leg portion 703. Still, in a more particular embodiment, the length 710 of the lower portion 705 is at least about 10%, such as at least about 15% of the length 713 of the leg portion 703. As such, according to one particular embodiment, the length 710 of the lower portion 705 is within a range between about 15% and about 50% of the length 713 of the leg portion 703.

In reference to certain dimensions, generally, the length 710 of the lower portion 705 is not greater than about 6 cm. In another embodiment, the length 710 is not greater than about 5 cm, or even not greater than about 4.5 cm. According to another embodiment, the length 710 of the lower portion 705 is at least about 2 cm, such as at least about 3 cm. As such, in one particular embodiment, the length 710 of the lower portion 705 is within a range between about 3 cm and about 4.5 cm.

The upper portion 706 has a length 712 that can be a fraction of the length 713 of the leg portion 703, measured between the distal end 704 and the opposing end at the articulating joint 718. The length 712 of the upper portion 706 is particularly designed to provide a suitable torque on an implant and lock the position of the upper portion 706 relative to the implant while other portions of the tool are manipulated. According to one embodiment, the length 712 of the upper portion 706 is not greater than about 80% of the length 713 of the leg portion 703. In another more particular embodiment, the length 712 of the upper portion 706 is not greater than about 75%, such as not greater than about 70% of the length 713 of the leg portion 703. Generally, the length 712 of the upper portion 706 is at least about 10%, such as at least about 20%, or even at least about 30% of the length 713 of the leg portion 703. In a particular embodiment, the length 712 of the upper portion 706 is within a range between about 30% and about 70% of the length 713 of the leg portion 703.

In reference to actual dimensions, in one embodiment, the length 712 of the upper portion 706 is not greater than about 7 cm. In a more particular embodiment, the length 712 of the upper portion 706 is not greater than about 6 cm, such as not greater than about 5.5 cm. In certain embodiments, the length 712 of the upper portion 706 is at least about 2 cm, such as at least about 3 cm. In a more particular embodiment, the length 712 of the upper portion 706 is within a range between about 3 cm and about 5.5 cm.

Notably, in certain embodiments the lower portion 705 has a length 710 that is different than the length 712 of the upper portion 706. For example, the lower portion 705 can have a length 710 that is less than the length 712 of the upper portion 706. In another embodiment, the length 710 of the lower portion 705 is not greater than about 95% of the length 712 of the upper portion 706. In another more particular embodiment, the lower portion 705 has a length 710 within a range between about 70% and about 95% of the length 712 of the upper portion 706. Notably, the dimensions of the lower portion 705 and upper portion 706 are particularly engineered to facilitate operation of the articulating joint and more particularly suitable locking of the leg portion 703 relative to an implant during a distraction or compression procedure.

FIG. 8 includes a side view of the portion 700 of the surgical tool illustrated in FIG. 7. As described previously, the portion 700 includes the proximal end 702, the distal end 704, the upper portion 706, and the lower portion 705 separated by the articulating joint 718. More readily illustrated in FIG. 8 is portion 801 which includes an opening at the proximal end 702 configured to engage a locking member (previously illustrated in FIG. 3) having teeth and configured to fixably engage portion 801 and lock the position of the members relative to each other.

FIG. 8 further illustrates the opening 720 within the pivot point 717 configured to engage an anchor 805, which can include a peg, screw or the like. This arrangement facilitates pivotally connecting the member 700 illustrated in FIG. 8 with the member 500 previously illustrated in FIG. 5. While the pivoting connection is illustrated as a hinged connection, it will be appreciated that other articulating connections can be used, for example a rod and clasp.

FIG. 8 further includes the articulating joint 718 including the opening 707 extending through a region of the lower portion 705 and the opening 709 extending through a region of the upper portion 706. According to the illustrated embodiment, the articulating joint 718 is a hinge, including a pin 803 configured to extend through openings 707 and 709 and pivotally connect the lower portion 705 and upper portion 706. In one embodiment, coupling of the upper portion 706 with lower portion 706 can include fitting a protrusion 809 of the upper portion 706 into a recess 807 of the lower portion 705 and aligning the openings 707 and 709 of these respective portions and fixing these portions relative to each other using the pin 803. While the particular embodiment of FIG. 8 illustrates a hinging mechanism as the articulating joint 718, it will be appreciated that other articulating joints can be used. Moreover, the articulating joint 718 can further include a locking mechanism, such as a ratcheting mechanism, configured to lock the movement of the upper portion 706 relative to the lower portion 705 and avoid movement in a direction towards a previous position until released by the operator.

FIG. 8 also more clearly details the head portion 719 adjacent to the distal end 704 of the upper portion 706. In particular, the head portion includes a channel 810 adjacent to the distal end 704 and configured to engage an implant, such as a rod. In one particular embodiment, during a distraction or compression procedure, the channel 810 can be connected to a rod and lock the upper portion 706 on the rod such that the position of the upper portion 706 is fixed relative to the rod.

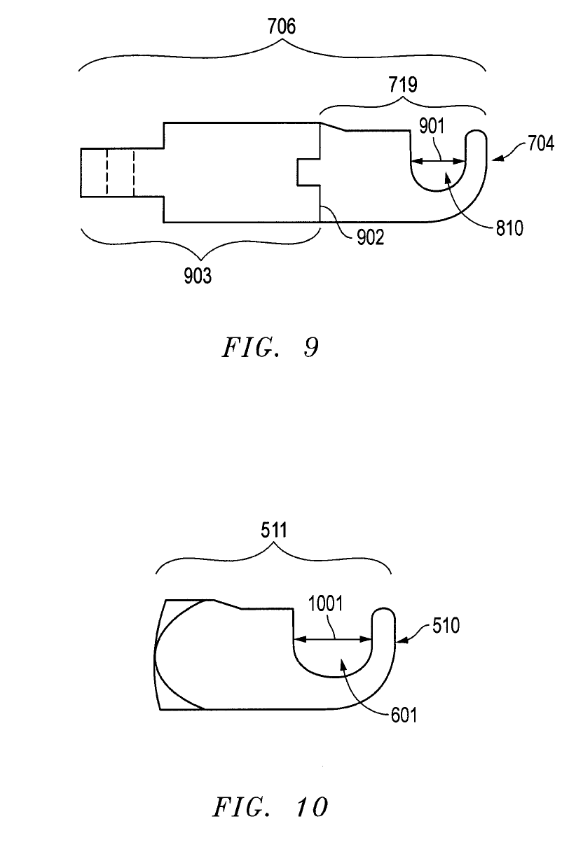

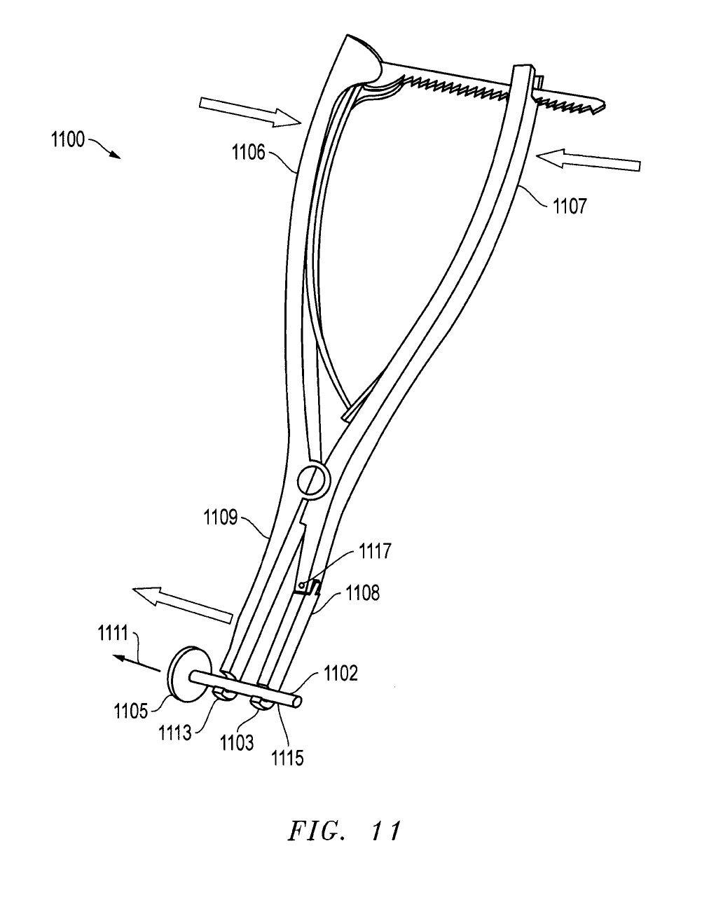

FIGS. 9 and 10 are described together to provide a comparison between particular features illustrated therein. FIG. 9 includes a side view of the upper portion 706, including the head portion 719, which includes a channel 810 adjacent to the distal end 704. FIG. 10 provides a side view of the head portion 511, including the channel 601 adjacent to the distal end 510. The channel 810 of the head portion 719 illustrated in FIG. 9 includes a width 901, measured as the greatest distance between parallel side walls. Moreover, the channel 601 of the head portion 511 illustrated in FIG. 10 also includes a width 1001, measured as the greatest distance between the parallel side walls of the channel 601.

Generally, the width 901 of the channel 810 is significantly different than the width 1001 of the channel 601, as the channel 810 is configured to fixably engage the rod and lock the upper portion 706 in a position on the rod, while the head portion containing the channel 601 is configured to slideably engage a rod and translate along the length of the rod relative to the head portion having the channel 810. As such, in one embodiment, the width 901 of the channel 810 is not greater than about 96% of the width 1001 of channel 601. In another embodiment, the width 901 is not greater than about 93%, such as not greater than about 90%, not greater than about 85%, or even not greater than about 80% of the width 1001 of the channel 601. Still, according to other embodiments, the width 901 of channel 810 is at least about 30%, such as at least about 40%, at least about 45%, or even at least about 50% of the width 1001 of channel 601. According to one particular embodiment, the width 901 of the channel 810 is within a range between about 50% and about 96% of the width 1001 of channel 601.

In reference to particular values, according to one embodiment, the width 901 of the channel 810 is at least about 2.50 mm. In another embodiment, the width 901 of the channel 810 is at least about 2.75 mm or even at least about 3.00 mm. Still, the width 901 of the channel 810 can be limited, such that in one embodiment, the width 901 of channel 810 is not greater than about 8.00 mm. In another embodiment, the width 901 of the channel 810 is not greater than about 7.50 mm, such as not greater than about 7.00 mm, or even not greater than about 6.75 mm. In certain embodiments, the width 901 is within a range between about 3.40 mm and 6.70 mm.

By way of comparison, the width 1001 of the channel 601 is generally at least about 5 mm. According to other embodiments, the width 1001 of the channel 601 is at least about 6 mm, such as at least 7 mm. However, the width 1001 of channel 601 is typically not greater than about 12 mm. In one embodiment, the width 1001 of the channel 601 is not greater than about 10 mm, or even not greater than about 8 mm. In a more particular embodiment, the width 1001 of channel 601 is within a range between about 7 mm and 8 mm. Notably, the range of widths 1001 for the channel 601 is less than the range of widths 901 of the channel 810 as the head portion 511 during a distraction or compression procedure is configured to slideably engage an implant, while the head portion 719 is configured to fixably engage the rod and fix the position of the upper portion 706 relative to the rod.

As such, the widths 901 and 1001 of the channels 810 and 610 respectively, can be different with respect to the rod each of the channels 810 and 610 are configured to engage. For example, in one embodiment, the width 901 of the channel 810 is greater than the diameter of the rod by not greater than about 20% of the diameter of the rod. In other certain embodiments, the width 901 of channel 810 is greater than the diameter of the rod by not greater than about 15%, such as not greater than about 12%, or even not greater than about 10% of the diameter of the rod. In one particular embodiment, the width 901 of the channel 810 is greater than the diameter of the rod within a range between about 3% and about 10% of the diameter of the rod. Notably, various rods exist within the industry, and each of these rods can have different diameters, however, the diameters typically range between about 3.50 mm and about 6.50 mm.

In reference to the channel 601, generally, the width 1001 of the channel 601 is greater than the diameter of the rod by at least 5% of the diameter of the rod. In another embodiment, the width 1001 is greater than the diameter of the rod by at least about 8%, such as at least about 10%, or least about 15% of the diameter of the rod. Typically, the width 1001 of channel 601 is greater than the diameter of the rod by not greater than about 60% of the diameter of the rod. In a more particular embodiment, the width 1001 of the channel 601 is greater than the diameter of the rod within a range between about 9% and about 55% of the diameter of the rod.

FIG. 9 further illustrates a quick-connect coupling 902 between the head portion 719 and a portion 903 of the upper portion 706. According to one embodiment, the leg portions of either of the members (illustrated in FIGS. 5 and 7) can include quick-connect couplings configured to engage a plurality of interchangeable heads attachable to the leg portions. According to one particular embodiment, only the upper portion 706 includes a quick-connect coupling 902 to engage a plurality of interchangeable heads, wherein each of the heads have a channel width 901 that is different than another interchangeable head. A plurality of interchangeable heads, each with different channel widths, facilitates engagement of the surgical tool with a variety of different rod diameters. According to one particular embodiment, the upper portion 706 includes a quick-connect coupling while the opposing leg portion does not include a quick-connect coupling, such a configuration may be suitable since the channel width of the head portion 719 must be a close fit to fixably engage the rod while the channel width 1001 of head portion 511 may be significantly larger than the rod, since it is configured to slideably engages the rod.

FIG. 11 illustrates a surgical tool 1100 during a distraction or compression procedure in accordance with one embodiment. During such procedures, the rod 1102 is engaged within the channels 1113 and 1115 and the operator can compress the members (i.e., the handles) 1106 and 1107 toward each other. The leg portion 1102 having the articulating joint 1117 is locked in position with respect to the rod 1102 independent of the movement of the members 1106 and 1107. The leg portion 1109 is dependent upon the movement of the members 1106 and 1107, and as such, during movement of the members 1106 and 1107 by the operator toward each other, the leg portion 1109 translates along the length of the rod 1102. The leg portion 1109 can slideably engage the rod 1102 and move in a direction along the rod 1102 away from leg portion 1108 and abut an implant 1105. Upon further translation of the leg portion 1109 along the longitudinal axis of the rod 1115, the leg portion 1109 can engage the implant 1105 and move the implant 1105 along the length of the rod 1102 as indicated by arrow 1111. The surgical tool disclosed herein obviates the need for a vise grip as the distraction or compression procedure is facilitated by the articulating joint 1117 and a channel 1115 configured to frictionally engage the rod 1102 and fix the position of the leg portion 1108 relative to the rod 1102 by exerting a torsional force on the rod 1102.

Accordingly, the surgical tool is well suited for manipulating implants, such as translating implants along a rod during a compression or distraction procedure. In more detail, the present surgical tool can be operated during a compression procedure such that upon manipulation of the surgical tool, implants connected to the rod can be translated along a portion of the rod and become closer together thereby locally compressing the spine in the region. In accordance with another embodiment, the surgical tool can also be used during a distraction procedure, wherein upon manipulation of the surgical tool as described herein, implants connected to the rod can be translated along a portion of the rod creating greater distance between the implants and thereby locally distracting the spine within the region.

Referring to FIG. 12, a perspective view of a surgical tool 1200 is provided according to another embodiment. The surgical tool 1200 includes members 1201 and 1202 (i.e., handles) pivotally connected together at a pivot point 1206 such that the members 1201 and 1202 can be moved between a first position and second position about a common axis defined by the pivot point 1206. As described in previous embodiments, a locking member 1203 is connected to the member 1201 and configured to interlock with the member 1202 during movement of the members 1201 and 1202 between a first position and a second position. Moreover, the surgical tool 1200 includes a spring 1204 extending between the members 1201 and 1202 and biasing the member 1201 against the member 1202. The spring 1204 facilitates inadvertent movement of the members 1201 and 1202 towards each other without operator intervention.

The surgical tool 1200 further includes leg portions 1205 and 1207 connected to the members 1201 and 1202, respectively. As illustrated, the leg portions 1205 and 1207 are connected to the members 1201 and 1202 and extend from the pivot point 1206. According to one embodiment, the members 1201 and 1202 can be fixably attached to leg portions 1205 and 1207. In another embodiment, the members 1201 and 1202 and leg portions 1205 and 1207 are unitary or monolithic pieces of material.

As illustrated, the leg portions 1205 and 1207 can be further connected to leg portions 1209 and 1211 respectively. In one embodiment, leg portions 1205 and 1207 are connected to leg portions by articulating joints 1228 and 1229 allowing for movement of the leg portions 1209 and 1211 relative to the leg portions 1205 and 1207. In a particular embodiment, the articulating joints 1228 and 1229 can be hinges, including for example engaging portions that fit together and are connected by a pin that facilitates a rotating connection around an axis defined by a pin.

The surgical tool 1200 can further include a member 1213 having arms 1214 and 1215 extending between and connected to the leg portions 1209 and 1211. In accordance with one embodiment, each of the arms 1214 and 1215 are connected to each of the leg portions 1209 and 1211. In one particular embodiment, and as further illustrated in FIG. 12, the member 1213 can include a plurality of arms, including a pair of arms 1213 and 1214 connected to a top side of the members 1209 and 1211 and another pair of arms 1235 and 1236 connected to a bottom side of the members 1209 and 1211. Notably, for such embodiments, the pairs of arms can be connected to each other.

Moreover, the arms 1214 and 1215 can be connected to each other, including for example by a connection 1231. In one particular embodiment, the connection 1231 can be a pivoting connection such that the arms 1214 and 1215 are moveable with respect to each other and allow portions of the arms 1214 and 1215 to slideably engage each other. The connection 1231 further facilitates relative motion between the arms 1214 and 1215 and the leg portions 1209 and 1211. In particular, the arms 1213 and 1214 facilitate maintaining a substantially parallel orientation between the leg portions 1209 and 1211 during manipulation of the members 1201 and 1202 between a first position and a second position.

In accordance with another embodiment, the arms 1214 and 1215 are connected to the leg portions 1209 and 1211 via connections 1232 and 1233 respectively at proximal ends of the arms 1214 and 1215. The connections 1232 and 1233 allow for rotation of the arms 1214 and 1215 with respect to the leg portions 1209 and 1207. As further illustrated in the embodiment of FIG. 12, the distal ends of the arms 1214 and 1215 are connected to the leg portions 1209 and 1211, respectively. In another embodiment, the distal end of each of the arms 1214 and 1215 can be connected to other leg portions 1209 and 1211, such that for example, the proximal end of the arm 1214 is connected to the leg portion 1209 and the distal end of the arm 1214 is connected to the leg portion 1211.

In accordance with another embodiment, the leg portions 1209 and 1211 include slots 1216 and 1217 for engagement and connection of the arms 1214 and 1215. For embodiments using pairs of arms along the top and bottom surface, such as the design illustrated in FIG. 12, the pairs of arms can be connected through the connections 1219 and 1221 allowing the same relative motion between the pairs of arms. In one particular embodiment, the members can include pins or the like, configured to slide within the slots 1216 and 1217 capable of translating the lengths of the slots 1216 and 1217. Accordingly, the connections 1219 and 1221 allow the arms 1214 and 1215 to move relative to the leg portions 1209 and 1211, and more particularly allow for angular displacement of the arms 1214 and 1215 relative to each other while maintaining a substantially parallel orientation between the leg portions 1209 and 1211.

As illustrated, the surgical tool 1200 further includes an articulating joint 1223 and an upper portion 1225 between the articulating joint 1223 and a distal end 1234. As described herein in accordance with other embodiments, the articulating joint 1223 can be a hinge or the like, facilitating relative independent motion of the upper portion 1225 to facilitate locking of the leg portion 1211 and member 1207 relative to an implant. Such a design allows for single-handed operation of the surgical tool which is suitable for imparting translational movement of an implant, and more particularly, well suited for completing distraction and/or compression procedures. It should be noted that while the member 1213 facilitates a substantially parallel orientation between the leg portions 1209 and 1211, during engagement of an implant, the upper portion 1225 is locked in a position that is not substantially parallel with the leg portion 1209.

According to one embodiment, the surgical tool and all of its components, including members, leg portions, head portions, and articulating joints are made of an autoclavable material. That is, a material capable of withstanding high temperatures and pressures used to sterilize surgical materials. As such, the surgical tool typically includes a metal, metal alloy, polymer, or ceramic material. According to one embodiment, suitable metal and metal alloys can include transition metals. In a particular embodiment, the surgical tool can include iron (e.g, steel), titanium, nickel, chromium, cobalt, tungsten, molybdenum, vanadium, and any combination thereof. Moreover, components of the surgical tool described herein can include ceramic materials, such that the entire component includes a ceramic, or more particularly the component includes a ceramic coating. In one embodiment, suitable ceramics can include oxides, carbides, borides, nitrides, or any combination thereof. Still, in one particular embodiment, the surgical tool includes oxides such as, silica, alumina, zirconia, titania, and the like, and any combinations thereof.

The surgical tool disclosed herein includes a combination of features previously unrecognized by state-of-the-art distraction pliers. Such features include an articulating joint present within one of the leg members facilitating locking of the position of the member relative to the rod, and channels within the head portions of the leg members having different widths with respect to each other, as well as with respect to the implant being engaged. Such features facilitate a distraction or compression procedure capable of being undertaken with a single hand and thus replacing the combination of tools typically used by surgeons including vise grips and conventional distraction pliers.

The above-disclosed subject matter is to be considered illustrative, and not restrictive, and the appended claims are intended to cover all such modifications, enhancements, and other embodiments that fall within the true scope of the present invention. For example, it is noted that the components in the exemplary embodiments described herein as having a particular function or as being located in a particular housing are illustrative and it is noted that such components can perform additional functions or be located in different configurations. Thus, to the maximum extent allowed by law, the scope of the present invention is to be determined by the broadest permissible interpretation of the following claims and their equivalents, and shall not be restricted or limited by the foregoing detailed description.

What is claimed is:

1. A surgical tool for manipulating an implant comprising:
a first member and a second member pivotally connected together at a pivot point and configured to be moved between a first position and a second position about a common axis defined by the pivot point; and
a first leg connected to the first member and a second leg connected to the second member, the first leg and the second leg extending from the pivot point and terminating at respective distal ends, wherein the first leg comprises a first portion that is displaceable between a first position and a second position independent of the position of the first member and the second member.

2. The surgical tool of claim 1, wherein the first leg comprises an articulating joint between the distal end and the pivot point.

3. The surgical tool of claim 2, wherein the articulating joint is a hinge, including a pin extending through an opening in the first portion.

4. The surgical tool of claim 2, wherein the first portion is between the articulating joint and the distal end.

5. The surgical tool of claim 4, wherein the first portion is configured to articulate around a single axis of the articulating joint in a direction towards the second leg.

6. The surgical tool of claim 2, wherein the first leg further comprises a second portion between the articulating joint and the pivot point.

7. The surgical tool of claim 6, wherein the second portion is configured to articulate around a single axis of the articulating joint in a direction away from the second leg.

8. The surgical tool of claim 6, wherein the second portion has a length that is not greater than about 80% of a length of the first leg.

9. The surgical tool of claim 1, wherein the first portion is displaceable between the first position and the second position at a displacement angle relative to the first portion and a second portion within the first leg of at least about 5°.

10. The surgical tool of claim 1, wherein the first portion has a length that is not greater than about 80% of a length of the first leg.

11. The surgical tool of claim 1, further comprising:
a first head connected to the first portion adjacent to the distal end of the first leg and configured to engage an implant; and
a second head connected to the second leg adjacent to the distal end of the second leg and configured to engage the implant.

12. The surgical tool of claim 11, wherein the first head comprises a first channel and the second head comprises a second channel.

13. The surgical tool of claim 12, wherein the first channel has a width that is less than a width of the second channel.

14. The surgical tool of claim 13, wherein the width of the first channel is not greater than 96% of the width of the second channel.

15. The surgical tool of claim 12, wherein the first channel has a width and is configured to engage a rod having a diameter, wherein the width of the first channel is greater than the diameter of the rod by not greater than about 20% of the diameter of the rod.

16. The surgical tool of claim 12, wherein the second channel has a width and is configured to slidably engage a rod having a diameter, wherein the width of the second channel is greater than the diameter of the rod by at least about 5% of the diameter of the rod.

17. A surgical tool configured to complete a distraction or compression procedure comprising:
a first member and a second member pivotally connected together at a pivot point and configured to be moved between a first position and a second position about a common axis defined by the pivot point; and
a first leg connected to the first member and extending from the pivot point to a distal end, and a second leg connected to the second member and extending from the pivot point to a distal end, wherein a first portion of the first leg is configured to engage a rod and fix the axial position of the first portion with respect to the rod during movement of the first member and second member between the first position and the second position.

18. The surgical tool of claim 17, wherein the second leg is displaced in a direction along the rod upon movement of the first member and second member between the first position and the second position.

19. The surgical tool of claim 17, wherein the first portion is configured to frictionally engage the rod and fix the position of the first portion relative to the rod by exerting a torsional force on the rod.

20. A surgical tool comprising:
a first member and a second member pivotally connected together at a pivot point, the first member comprising:
a proximal end and a distal end, wherein the pivot point is between the proximal end and the distal end, and a first channel between the distal end and pivot point; and
the second member comprising:
a proximal end and a distal end, wherein the pivot point is between the proximal end and the distal end, and a second channel between the pivot point and the distal end, wherein the second channel has a width that is greater than a width of the first channel.

21. A surgical tool for performing a distraction or compression of implants comprising:
a first member having a first proximal portion between a proximal end and a pivot point and a first distal portion between a distal end and the pivot point; and
a second member pivotally connected to the first member at the pivot point and having a second proximal portion between a proximal end and a pivot point and a second distal portion between a distal end and the pivot point, wherein the second distal portion comprises at least one more articulating joint than the first distal portion.

22. The surgical tool of claim 21, further comprising a third member comprising the first arm and the second arm, wherein the first arm and the second arm are pivotally connected to the first distal portion of the first member and the second distal portion of the second member.

23. The surgical tool of claim 20, wherein the first arm and the second arm are connected to each other.

24. The surgical tool of claim 21, further comprising a fourth member, comprising a third arm and a second arm, wherein the third arm and the fourth arm are pivotally connected to the first distal portion of the first member and the second distal portion of the second member.

25. The surgical tool of claim 24, the third member and the forth member are connected to each other.
